(19) United States
(12) Patent Application Publication
(10) Pub. No.: US 2004/0193201 A1
(43) Pub. Date: Sep. 30, 2004

(54) Disposable lancing device

(76) Inventor: Yong Pil Kim, Seoul (KR)

Correspondence Address: MERCHANT & GOULD PC, P.O. BOX 2903, MINNEAPOLIS, MN, 55402-0903, US

(21) Appl. No.: 10/409,738

(22) Filed: Apr. 9, 2003

(30) Foreign Application Priority Data
Mar. 24, 2003 (KR) 2003-8753

(51) Int. Cl. A61B 17/32

(52) U.S. Cl. 606/181

(57) ABSTRACT

A disposable lancing device is disclosed, which may not return to its initial state after projection of a lancet, thereby preventing infectious possibility due to voluntary reuse of the device.

The disclosed disposable lancing device for injecting a lancet into the skin by elasticity stored in an elastic member to obtain blood, comprising: an operating member having a lancet attached to the fore portion, and an elastic member connected to the rear portion; a body receiving the operating member and the elastic member; a pushing member maintaining the operating member with the elasticity stored in the elastic member; and a retreat-preventing means fixing the operating member not to move backward again after ejection.

### BACKGROUND OF THE INVENTION

1. Field of the Invention

The present invention relates to a disposable lancing device to withdraw a blood sample for treatment, and more specifically, to a disposable lancing device which may not be reused after a lancet is projected.

2. Description of the Prior Art

Generally, a lancing device is used when a small amount of blood is withdrawn for blood type test or glycosuria examination in blood of diabetes while an injector is used when a large quantity of blood is withdrawn.

FIGS. 1 and 2 are perspective and cross-sectional views of a conventional disposable lancing device. As shown in FIGS. 1 and 2, a lancet 35 is hold at the fore portion of an operating member 30, and an elastic member 40 is engaged with the rear portion of the operating member 30. The operating member 30 has a protruding portion 32 at its rear portion. The protruding portion 32 is fixed by a lower end sloping side 22 which is inserted toward the inside of a body 10 and formed having a single unit with a pushing member 20 attached to the outside of the body 10. When the pushing member 20 is pushed, the sloping side 22 is tilted to release the fixed operating member 30, and then the operating member 30 is sprung toward due to elasticity stored in the elastic member 40. As a result, the lancet 35 hold at the fore portion of the operating member 30 is shot from the body 10. However, in the conventional disposable lancing device, if a user pushes the operating member 30 backward voluntarily, the sloping side 22 of the pushing member 20 can be pushed by the protruding portion 32 of the operating member 30, and the protruding portion 32 can return to its initial state. Thereafter, the sloping side 22 of the pushing member 20 also returns to the initial state due to elasticity stored in the spring member 40 such that the operating member 30 is fixed to the pushing member 30 with the same elasticity as the initial state. Accordingly, although it is easy for the user to reuse the disposable lancing device voluntarily, the used lancing device has sanitary problems such as infection.

### SUMMARY OF THE INVENTION

In order to overcome the above-described problem, the present invention has an object to provide a disposable lancing device, which may not return to its initial state after projection of a lancet, thereby preventing infectious possibility.

The present invention has another object to provide a disposable lancing device, which can be easily assembled due to the reduced number of components and conveniently used due to its controllable ejection length of the lancet.

In order to accomplish the above-described objects, there is provided a disposable lancing device for injecting a lancet into the skin by elasticity stored in an elastic member to obtain blood, comprising: an operating member having a lancet attached to the fore portion, and an elastic member connected to the rear portion; a body receiving the operating member and the elastic member; a pushing member maintaining the operating member with the elasticity stored in the elastic member; and a retreat-preventing means fixing the operating member not to move backward again after ejection.

The retreat-preventing means comprises a wing plate formed on a certain portion of the operating member, and a sloping plate formed in the body parallel with the wing plate, wherein the wing plate is placed at the rear of the sloping plate when elasticity is stored in the elastic member due to retreat of the operating member, and the wing plate is interlocked by the sloping plate after ejection of the operating member, thereby preventing the operating member from moving backward.

The wing plate is formed aslant such that the end portion of the wing plate is toward the rear portion of the body, and the sloping plate is formed aslant such that the end portion of the sloping plate is toward the fore portion of the body.

A cap for ushering ejection of the lancet and regulating the gap between the body and the cap by rotation is attached to the fore portion of the body, wherein the gap between the cap and the body is regulated by movement of a helical internal thread formed in the cap according to a helical groove formed in the body.

### BRIEF DESCRIPTION OF THE DRAWINGS

The present invention will be explained in terms of exemplary embodiments described in detail with reference to the accompanying drawings, which are given only by way of illustration and thus are not limitative of the present invention, wherein:

FIG. 1 is a perspective view of a conventional disposable lancing device;

FIG. 2 is a cross-sectional view of the conventional disposable lancing device;

FIG. 3a is a perspective view of a disposable lancing device according to the present invention;

FIG. 3b is a exploded perspective view of the disposable lancing device according to the present invention;

FIG. 3c is a cross-sectional view of the disposable lancing device according to the present invention;

FIG. 4a is a cross-sectional view of the disposable lancing device according to the present invention, wherein a cap adheres closely to a body;

FIG. 4b is a cross-sectional view of the disposable lancing device according to the present invention, wherein the cap is detached from the body;

FIG. 5a is a cross-sectional view of the assembled disposable lancing device according to the present invention;

FIG. 5b is a cross-sectional view of the disposable lancing device according to the present invention, wherein an operation member is fixed backward;

FIG. 5c is a cross-sectional view of the disposable lancing device according to the present invention, wherein a wing plate passes a sloping plate;

FIG. 5d is a cross-sectional view of the disposable lancing device according to the present invention, wherein a lancet is ejected to the utmost;

FIG. 5e is a cross-sectional view of the disposable lancing device according to the present invention, wherein the wing plate is interlocked by the sloping plate; and

FIG. 6 is a cross-sectional view of another example of a disposable lancing device according to the present invention.

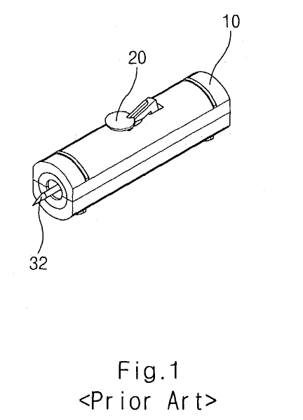

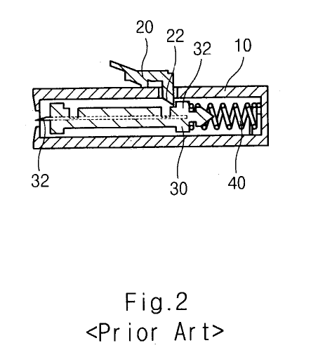

### DETAILED DESCRIPTION OF THE PREFERRED EMBODIMENTS

The present invention will be described in detail with reference to the accompanying drawings.

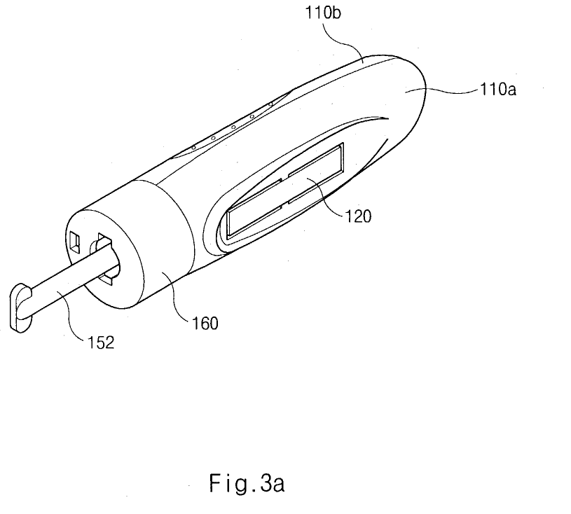

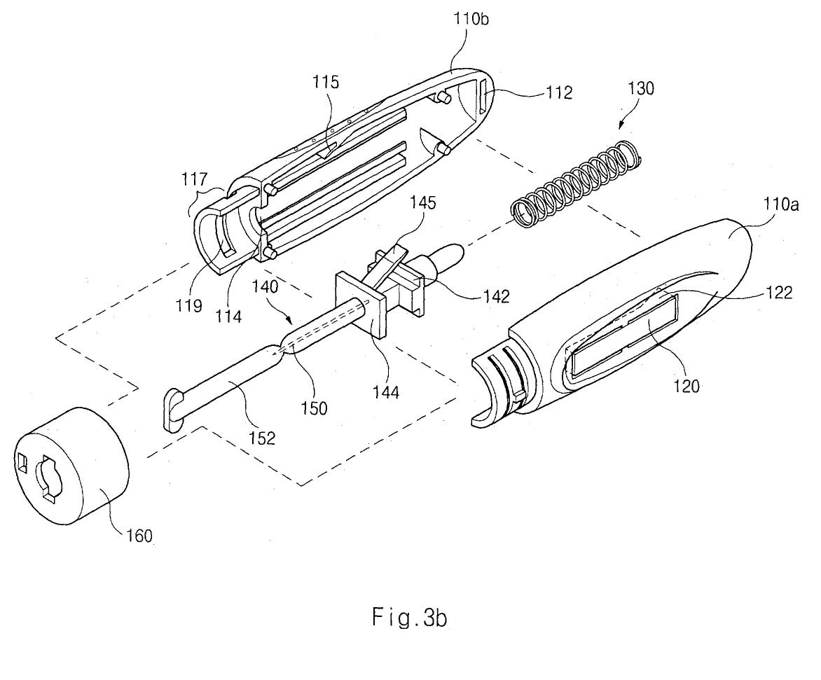

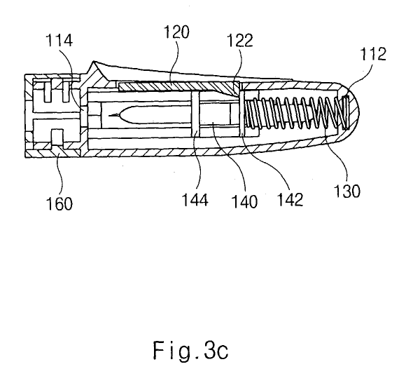

FIGS. 3a, 3b and 3c are perspective, exploded perspective and cross-sectional views of a disposable lancing device according to the present invention.

As shown in FIGS. 3a, 3b and 3c, the disclosed disposable lancing device comprises an operating member 140, a pushing member 120, bodies 110a and 110b and a cap 160. The operating member 140 has a lancet 150 attached to its fore portion and an elastic member 130 combined with its rear portion. The pushing member 120 fixes the operating member 140 with the elasticity stored in the elastic member 130 while the lancing device is not used. The bodies 110a and 110b equipped with the pushing member 120 receive the operating member 140 and the elastic member 130 therein. The bodies 110a and 110b have the cap 160 at the fore portion (at the left side of Figs.) for ushering the ejection of the lancet 150 and regulating the gap between the bodies 110a and 110b and the cap by rotation.

The operating member 140 is formed to move forward and backward within the bodies 110a and 110b. The end of the elastic member 130, which is attached to the rear portion of the operating member 140, is fixed to a fixing groove 112 of the body. If the operating member 140 is pushed into the body, elasticity is stored in the elastic member 130. In order to maintain this state, a protruding portion 142 formed on a certain portion of the operating member 140 is fixed by a latch 122 of the pushing member 120.

The pushing member 120 formed in the body 110a has the wedge-shaped latch 122 to the rear portion. As the operating member 140 moves backward, and the protruding portion 142 passes the latch 122, the elastic member 120 is retracted. In this state, when the latch of the pushing member 120 is pressed down, the latch 122 is latched onto the protruding portion 142 at its retreat state. That is, the operating member 140 is fixed with elasticity stored in the elastic member 130.

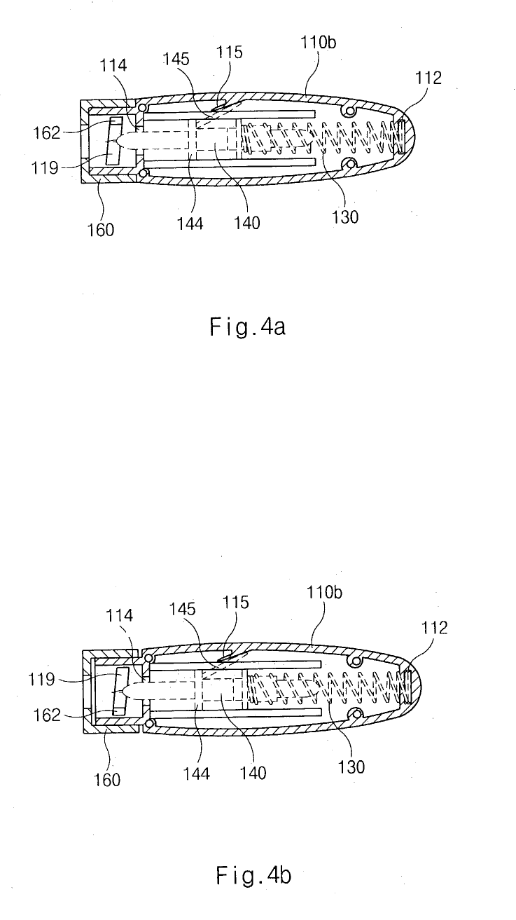

A wing plate 145 formed aslant on a certain portion of the operating member 140, and a sloping plate 115 formed in the body parallel with the wing plate 145 serve for prevention of reuse of the disclosed lancing device. The operating principles of the wing plate 145 and the sloping plate 115 will be explained referring to FIGS. 4a to 4e.

A stop projection 144 is formed on the fore portion of the operating member 140 to which the lancet 150 is attached. Since the stop projection 144 cannot pass through a hole 114 of the body, when the operating member 140 is moved forward by elasticity and inertia force, the ejected length of the lancet can be maintained at its regular length.

However, since the disclosed lancing device comprises the cap 160 for regulating the gap between the cap 160 and the body, the ejected length of the lancet 150 to pierce the skin can be changed by regulating the cap 160.

FIG. 4a is a cross-sectional view of the disposable lancing device according to the present invention, wherein the cap adheres closely to the body, and FIG. 4b is a cross-sectional view of the disposable lancing device according to the present invention, wherein the cap is detached from the body.

In order to regulate the ejected length of the lancet to pierce the skin, as shown in FIG. 3b, a neck portion 117 formed in the fore portion of the bodies 110a and 110b has a helical groove 119 on its surface. When the cap 160 is fit into the neck portion 117, a helical internal thread 162 formed in the cap 160 is screwed into the helical groove 119 formed in the neck portion 117. The helical internal thread 162 moves according to the helical groove 119 by rotation of the cap 160. As a result, the gap between the cap 160 and the body can be regulated. As shown in FIG. 4b, if the cap becomes more distant from the body, the inserted length of the lancet 150 (the ejected length of the lancet from the cap) becomes shorter. However, as shown in FIG. 4a, if the cap adheres closely to the body, the inserted length of the lancet 150 becomes longer.

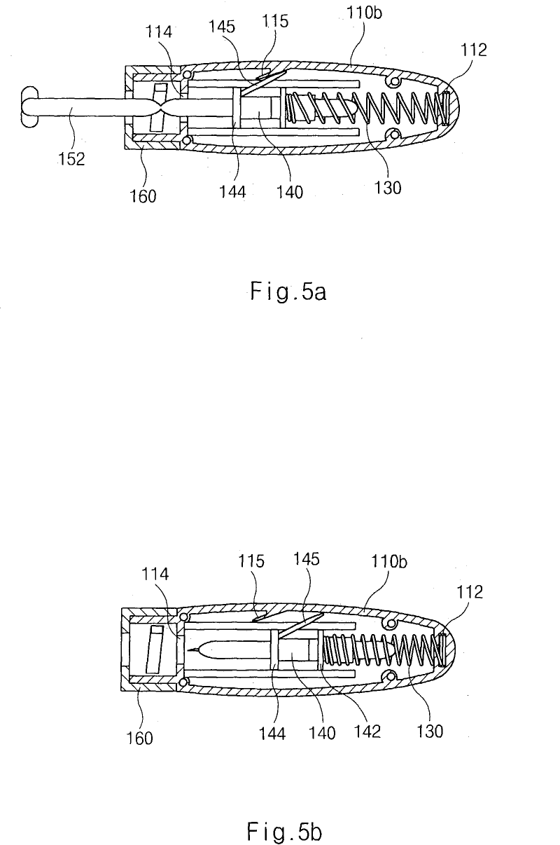

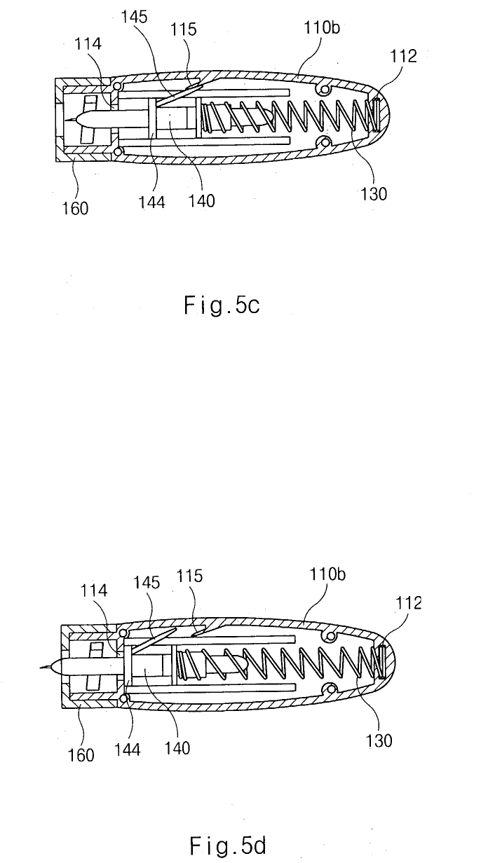

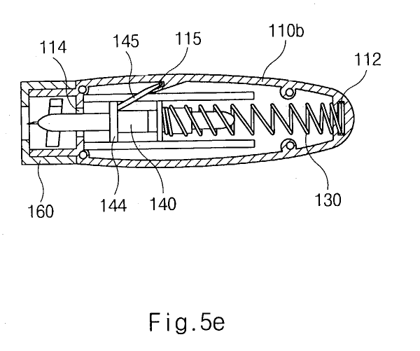

FIGS. 5a to 5e are views illustrating the operating principle of the disposable lancing device according to the present invention. FIG. 5a is a cross-sectional view of the assembled disposable lancing device. FIG. 5b is a cross-sectional view of the disposable lancing device, wherein an operation member is fixed backward. FIG. 5c is a cross-sectional view of the disposable lancing device, wherein a wing plate passes a sloping plate. FIG. 5d is a cross-sectional view of the disposable lancing device, wherein a lancet is ejected to the utmost. FIG. 5e is a cross-sectional view of the disposable lancing device, wherein the wing plate is interlocked by the sloping plate.

Referring to FIGS. 5a to 5e, if the elastic member 130 is put into the operating member 140, and the end portion of the elastic member 130 is fixed at the fixing groove 112 of the body, the wing plate 145 is located at the rear portion of the sloping plate 115 formed in the body. In this state, the disclosed disposable lancing device is assembled (see FIG. 5a). When a user uses the disclosed disposable lancing device, the user pushes a protecting cap 152 connected to the fore of the operating member 140, and then the operating member 140 is pushed into the body. In this state, as described in FIG. 3c, if the user presses down the rear portion of the pushing member 120, the protruding portion 142 of the operating member 140 is locked by the latch 122 of the pushing member 120 such that the operating member 140 is fixed backward (see FIG. 5b). In this state, if the user removes the protecting cap 152 and pushes the fore portion of the pushing member 120, the locking between the operating member 140 and the pushing member 120 is released such that the operating member 140 and the lancet 150 is ejected by the expansion of the elastic member 130.

Under the same state as FIG. 5b, the wing plate 145 is located to the rear end portion of the sloping plate 115. When the pushing member 120 is pushed, the operating member 140 is protruded toward due to elasticity and inertia force of the elastic member 130. In this process, because the operating member 140 passes the sloping plate 115 with the end of the wing plate 140 bent down (see FIG. 5c), the operating member 140 does not receive any resistance but moves until the stop projection 144 touches the internal wall of the hole 144 (see FIG. 5d). When the stop projection 144 touches the internal wall of the hole 144, the inertia force is removed and the elastic member 130 is re-contracted, thereby retreating the operating member 140. Since the wing plate 145 is restored into the original shape when the operating member 140 is retreated is interlocked by the sloping plate 115. As a result, the operating member 140 is no longer retreated due to the wing plate 145 interlocked by the sloping plate 115, as shown in FIG. 5e. Although a user tries to retreat the operating member in this state, the user cannot retreat it because the wing plate 145 is interlocked by the sloping plate 115. As a result, the disclosed lancing device cannot be reused by any user voluntarily.

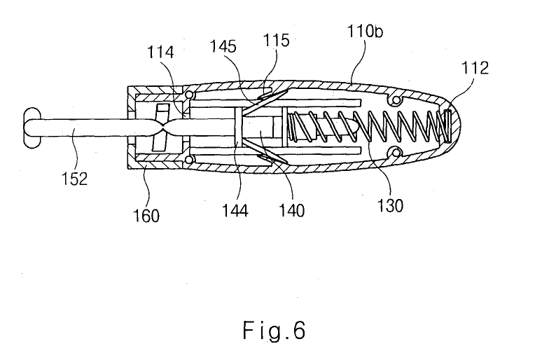

FIG. 6 is a cross-sectional view of another example of the disposable lancing device according to the present invention. As shown in FIG. 6, if the disclosed lancing device comprises two or more wing plates 145 and corresponding sloping plate 115, it can have more powerful interlocking force between them.

As discussed earlier, the disclosed disposable lancing device comprises the operating member which cannot be put into the body after ejection of the lancet because the wing plate formed on the operating member is placed at the fore portion of the sloping plate formed in the body. Accordingly, a user cannot put the lancet into the device voluntarily, thereby preventing the reuse of the lancing device.

Additionally, the disclosed disposable lancing device is configured to load a spring after assembly and include the pushing member formed in the body, thereby having an excellent assembling characteristic.

What is claimed is:

1. A disposable lancing device for injecting a lancet into the skin by elasticity stored in an elastic member to obtain blood, comprising:
an operating member having a lancet attached to the fore portion, and an elastic member connected to the rear portion;
a body receiving said operating member and said elastic member;
a pushing member maintaining said operating member with the elasticity stored in said elastic member; and
a retreat-preventing means fixing said operating member not to move backward again after ejection.

2. The disposable lancing device of claim 1, wherein said retreat-preventing means comprises a wing plate formed on a certain portion of said operating member, and a sloping plate formed in the body parallel with the wing plate,
wherein said wing plate is placed at the rear of said sloping plate when elasticity is stored in said elastic member due to retreat of said operating member, and said wing plate is interlocked by said sloping plate after ejection of said operating member, thereby preventing said operating member from moving backward.

3. The disposable lancing device of claim 2, wherein said wing plate is formed aslant such that the end portion of said wing plate is toward the rear portion of said body, and said sloping plate is formed aslant such that the end portion of said sloping plate is toward the fore portion of said body.

4. The disposable lancing device of claim 3, wherein said wing plate and said sloping plate are at least one or more.

5. The disposable lancing device of claim 1, wherein a cap for ushering ejection of the lancet and regulating the gap between the body and said cap by rotation is attached to the fore portion of the body.

6. The disposable lancing device of claim 5, wherein the gap between said cap and said body is regulated by movement of a helical internal thread formed in said cap according to a helical groove formed in said body.

7. The disposable lancing device of claim 4, wherein a cap for ushering ejection of the lancet and regulating the gap between the body and said cap by rotation is attached to the fore portion of the body.

8. The disposable lancing device of claim 7, wherein the gap between said cap and said body is regulated by movement of a helical internal thread formed in said cap according to a helical groove formed in said body.
